(19) United States
(12) Patent Application Publication
(10) Pub. No.: US 2009/0292314 A1
(43) Pub. Date: Nov. 26, 2009

(54) LUMBAR INTERSPINOUS PROSTHESIS AND ITS APPLICATIONS

(75) Inventors: Paolo Mangione, Pessac (FR); Jerome Levieux, Bellevue (CH)

Correspondence Address: YOUNG & THOMPSON, 209 Madison Street, Suite 500, ALEXANDRIA, VA, 22314, US

(73) Assignee: SPINEART SA, Geneve (CH)

(21) Appl. No.: 12/441,623

(22) PCT Filed: Sep. 14, 2007
(86) PCT No.: PCT/FR2007/051938

§ 371 Date: Mar. 17, 2009

(30) Foreign Application Priority Data
Sep. 18, 2006 (FR) 06 08155

(51) Int. Cl. A61B 17/70; A61F 2/44

(52) U.S. Cl. 606/249; 623/17.11

(57) ABSTRACT

A lumbar inter-spine prosthesis includes a torsion spring with two branches (3, 4) each end of which includes a fixation system (5) for attachment to the apophysis (1) of a vertebra (2).

The present invention relates to a lumbar interspinous prosthesis and to its applications.

Surgical treatment of degenerative disease has expanded greatly over the course of the last two decades. In addition to the procedures performed to provide simple relief of pressure on the nerve root (hernia ablation, diskectomy, recalibration) and the procedures involving vertebral osteosynthesis, there have been more recent developments in what are called non-fusion techniques, either taking the form of prosthetic disks implanted by an anterior approach, or taking the form of posterior implants that provide a certain degree of stabilization but still permit mobility.

Some of these implants afford this stabilization by a mechanism comprising an interspinous wedge, which especially limits extension, which may or may not be associated with a ligament surrounding the spinous process, and which limits flexion (interspinous wedges are marketed by Abbottspine under the name Wallis® or by Medtronic under the name Diam®.

Although these implants have given clinically satisfactory results, they do not restore normal kinetics to the segment that has been stabilized.

An interspinous wedge made of titanium and formed in a U shape is already known (see FR-A-2 722 980). This wedge allows the intervertebral space to be stabilized and thus reduces aging of the disk. However, the rigidity of this system is much too great to permit harmonious restoration of the movements of the lumbar spine.

Moreover, the anchoring is on one side of the spinous process, which results in less effective functioning during flexion and unreliable stability in the space.

Finally, as this system forces the surgeon to access the spinous process from both sides, it is necessary to detach muscles within a healthy region in order to be able to fit the implant in place. Most of the time, in fact, root compression is unilateral. The surgeon therefore makes his approach only from one side. However, in order to fit a device of this type in place, he will be forced to approach from both sides, which will increase the destabilization of the segment of the spine. Moreover, it is very disagreeable for a surgeon to access a healthy region in order simply to fit a device in place.

It would therefore be desirable to have a device allowing the lumbar segment to be stabilized while at the same time restoring kinetics similar to the physiological kinetics, that is to say comprising a center of rotation situated a little below the lower vertebral plateau of the segment being operated on, in its rear part. It would also be desirable for it to be able to be fitted in place by approaching the segment from only one side of the apophysis.

After extensive research, the Applicant has developed a novel type of lumbar interspinous prosthesis comprising a torsion spring (a spring of the “clothes peg” type working on itself in rotation).

It is for this reason that the present invention relates to a lumbar interspinous prosthesis which is characterized in that it comprises a torsion spring with two branches, each end of which has a system for fixation to an apophysis of a vertebra.

Although the spring used can have non-contiguous turns, it is preferable for the turns to be contiguous.

The branches of the torsion spring are designed to be fixed to apophyses of two adjacent vertebrae, in particular to the spinous processes, especially of the lumbar vertebrae, the axis of the spring being offset with respect to the straight line joining the centers of the systems for fixation to the apophyses.

The ends of the branches of the spring can be fastened directly to the apophyses, or in particular indirectly by way of a component which fixes itself to the apophyses.

For direct fixation, the ends of the branches of the spring can be simply provided with a loop. For example, the end of each branch folds back in a loop shape in order to form a hook (or clamp). In a variant embodiment, the loop can cooperate with a screw fitted in the apophysis.

For indirect fixation, the ends are equipped with a component for fastening to the apophysis without drilling. This fastening component is in particular a clip.

The clip comprises, for example, a generally flat elongate blade whose ends are curved on the same side in order to form a clamp for fastening to the apophysis for which it is intended. The space between the curved parts is adapted to the size of the apophysis. The elongate blade preferably has a Y shape in order to form two hooks at one end and a single hook at the other end.

The blade can be made up of several components. In particular, it comprises two sliding parts, one situated toward the turns of the spring and referred to as “inner”, and one situated remote from the turns of the spring and referred to as “outer”. Thus, in order to fit it in place, it is possible to lengthen the outer blade, for example, by sliding, then bring the hooks of each of the two sliding parts closer so as to clamp the apophysis. The clamp thus formed can then be blocked. To do so, the outer sliding part, for example, can comprise an elongate longitudinal slot, while the inner sliding part can comprise a threaded pin for guiding and blocking the clamp on the apophysis by cooperation with a nut or a thread formed in the inner sliding part, or vice versa. One branch can also constitute a slide for the other branch. For this purpose, its longitudinal edges can be curved in order to form an open channel for the other branch.

The cooperation between the spring and the device for fastening to the apophysis without drilling can be achieved by any known means, preferably permitting relative rotation of the two components. Under preferred conditions of use, the face of the elongate blade directed away from the face forming the hook comprises a stud that permits fixation of the end of one branch of the spring, for example by way of a loop provided at this end, leaving free the rotation. The stud is advantageously provided with a shoulder, so as to sandwich a branch of the spring between said shoulder and the elongate blade. Conversely, the stud can be provided with a groove in which is fitted a loop provided at the end of a branch of the spring.

Under other preferred conditions of implementation of the invention, one of the branches of the spring is folded a first time parallel to the axis of the spring, at the end point of the turns, and is folded a second time at the other end of the turns, at a right angle, such that the two branches are in the same plane and the axis of the spring is perpendicular to this plane.

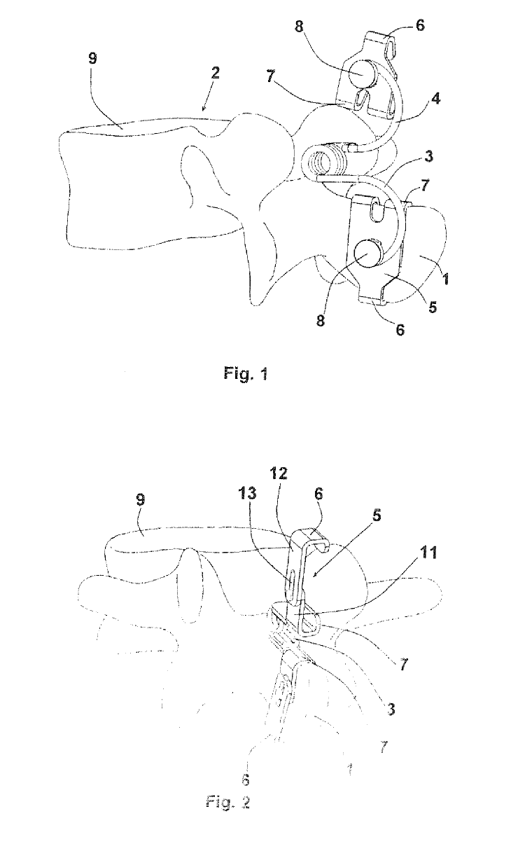

Under yet other preferred conditions of implementation of the invention, the two branches are curved along more than 50% of their length, as is illustrated in FIG. 1, or are curved in a hairpin shape, as is illustrated in FIG. 2.

Generally, the spring will be made of round wire. It will also be made in particular of metal suitable for implantation. It is preferably made of titanium or of titanium alloy. The possible other elements of the interspinous prosthesis are advantageously made of the same materials.

The external diameter of the spring is advantageously 3 to 13 mm, preferably 4 to 12 mm, particularly 5 to 11 mm, more particularly 6 to 10 mm.

The diameter of the wire from which the spring is made is advantageously 1.0 to 4.5 mm, preferably 1.0 to 4.0 mm, particularly 1.5 to 3.5 mm, more particularly 2 to 3 mm.

The axis of the spring, at rest, is offset with respect to the straight line joining the centers of the outer hooks for anchoring on the apophyses preferably by 3 to 18 mm, particularly by 4 to 16 mm, more particularly by 5 to 14 mm.

The interspinous prostheses forming the subject matter of the present invention have very useful properties and qualities. They are in particular capable of restoring, to the segment of the vertebral column that has been operated on, kinetics similar to the physiological kinetics, that is to say a center of rotation situated a little below the vertebral plateau of the lower vertebra of the segment operated on, and in its posterior part.

When the spring is compressed, the hooks can be placed between the spinous processes by a unilateral approach. The surgical procedure is thus made easier. Moreover, this avoids the surgeon accessing the column from both sides of the spinous process, thereby limiting the destabilization of the segment and leaving a healthy region intact.

The movement of the spring has an instantaneous center of rotation close to that of a healthy person (under the plateau of the lower vertebra, in the posterior third).

The flexibility of the type of spring used permits an easy and harmonious movement.

The clips with optional hooks permit reliable anchoring and function both in extension and in flexion.

These properties are illustrated below in the experimental section. They justify the use of the above-described interspinous prostheses in the treatment of degenerative diseases of the lumbar vertebrae.

It is for this reason that the present application also relates to a method for stabilizing a lumbar articulation, characterized in that an interspinous prosthesis of the above type is fitted in place between two vertebrae, preferably two lumbar vertebrae.

In particular, the branches of the spring are brought together with the aid of a suitable ancillary instrument, the ends of the branches are fixed to the apophyses, in particular the spinous processes, of two adjacent vertebrae, and the branches are released.

The preferred conditions of use of the above-described interspinous prostheses apply also to the other subjects of the invention mentioned above, in particular to the methods for stabilization of a lumbar articulation.

The invention will be better understood by referring to the attached drawings, in which:

FIG. 1 shows a side view of an interspinous prosthesis equipped with clips and mounted on a lumbar vertebra,

FIG. 2 shows a front elevation of another interspinous prosthesis equipped with clips and mounted on a lumbar vertebra,

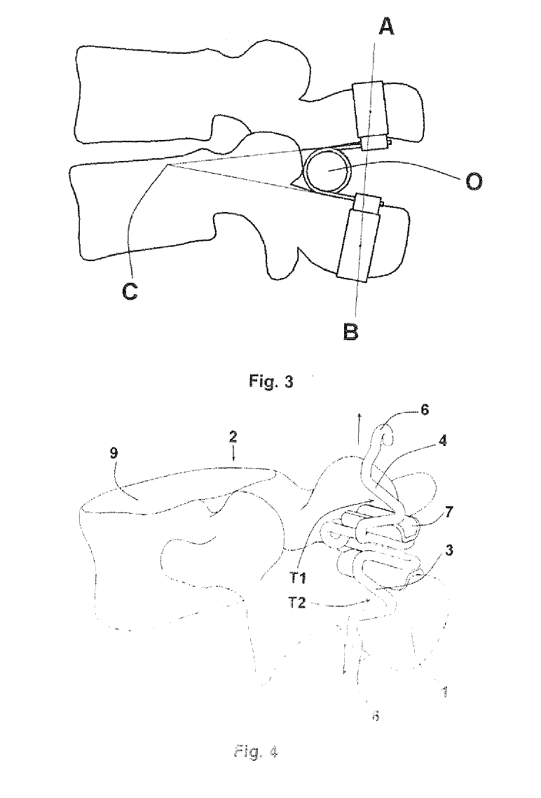

FIG. 3 shows a side elevation of an interspinous prosthesis equipped with clips and mounted on two lumbar vertebrae, schematically illustrating the axis of rotation of the prosthesis, and

FIG. 4 shows a side elevation of a variant interspinous prosthesis in which the ends of the branches of the spring are fastened directly to the apophyses.

An interspinous prosthesis according to the invention can be seen in FIG. 1, equipped with clips and mounted on the spinous process 1 of a lumbar vertebra 2.

The interspinous prosthesis shown comprises a torsion spring with two branches 3 and 4. The spring used has contiguous turns. Each of the ends of the two branches 3 and 4 comprises a system for fixation to an apophysis of a vertebra, which system is a clip 5. The clip 5 comprises a generally flat elongate blade whose ends are curved on the same side in order to form a hook 6, 7 for fastening to the apophysis 1. The space between the curved parts 6, 7 is adapted to the size of the apophysis. The elongate blade has a Y shape in order to form two hooks 7 at one end and a single hook 6 at the other end.

The upper branch 4 of the spring is folded a first time parallel to the axis of the spring, to the left, at the end point of the turns, and is folded a second time at the other end of the turns, to the right and at a right angle, such that the two branches 3, 4 are situated substantially in the same plane and the axis of the spring is perpendicular to this plane.

The face of the elongate blade directed away from the face forming the hook comprises a stud 8 that permits fixation of the end of one branch 3, 4 of the spring, by way of a loop provided at this end. This loop leaves free the relative rotation between the two components.

It will be seen that the axis of the spring is spaced apart from the straight line joining the center of the hooks 6 (offset relative to the latter).

The two branches 3, 4 are curved in an arc of a circle, almost starting from the turns of the spring, along more than 50% of their length.

The branches and the hooks are designed in such a way that the spring is located in a position under the vertebral plateau 9. Thus, the interspinous prosthesis according to the invention has a center of rotation situated a little below the vertebral plateau 9 of the lower vertebra 2 of the segment operated on, and in its posterior part (the upper vertebra on which the second clip is fastened is not shown).

The studs 8 are provided with a shoulder directed toward the front in the figure, so as to sandwich the branches 3, 4 of the spring between said shoulder and the elongate blade of the clip 5. The elongate blade, on the face directed away from the stud 8, is folded to the right in order to form hooks 6, 7, one hook 6 being situated at the top and two hooks 7 being situated at the bottom for the upper clip, and the opposite for the lower clip.

FIG. 2 shows a variant of the invention. To avoid having to fold the hooks onto the apophyses, the clip 5 also comprises a generally flat elongate blade whose ends are curved on the same side in order to form a hook 6, 7 for fastening to the apophysis 1, but the blade is made up of several components.

It comprises two sliding parts, namely an inner part 11 and an outer part 12. Thus, in order to fit it in place, the clip 5 can be lengthened by sliding the outer part of the blade, then bringing the hooks of each of the two sliding parts closer so as to clamp the apophysis. The clamp thus formed can then be blocked. The outer sliding part 12 comprises an elongate longitudinal slot 13, while the inner part 11 comprises a threaded pin for guiding and blocking the clamp on the apophysis 1 by cooperation with a thread provided in the inner part 11 of the blade. The screwing of the threaded pin in the inner part 11 of the blade blocks the sliding movement and firmly secures the clip 5 to the apophysis 1.

In this variant, the two branches 3 and 4 of the spring are welded to the inner part 11 of the clip 5.

As will be seen from FIG. 3, the spring has an instantaneous center of rotation C close to that of a healthy person (under the plateau of the lower vertebra, in the posterior third). In addition, the axis 0 of the spring is offset with respect to the straight line AB joining the centers of the outer hooks for fixation of the prosthesis on the apophyses.

FIG. 4 shows basically the same elements as in FIGS. 1 and 2. In this variant, at the end point of the spring, the branches 3 and 4 are folded in a hairpin and comprise a component in the form of an elongate trough that serves as the first and inner hook 7. In this variant, this component has been welded onto the branches in the area of the hairpin. The branches continue outward, initially forming a V shape (thus forming 2 open triangles T1 and T2), of which the outer end of the branch is folded in a loop shape, parallel to the axis of the spring, in order to form the second and outer hook 6. The V-shaped parts of the branches are substantially in the same plane, said plane being perpendicular to the axis of the spring.

In this variant, the branches 3 and 4, being folded in a V shape, have a length that gives them sufficient elasticity to allow the hooks 6 and 7 to be moved away from each other (as is shown by the two vertical arrows). A clamp can be used for this purpose. The release of the clamp permits secure fixation of the prosthesis to the apophyses 1. It goes without saying that a curved shape of the branches 3 and 4, as is shown in FIG. 1, is also suitable.

1-11. (canceled)

12. A lumbar interspinous prosthesis, comprising a torsion spring, having an axis (0) and an external diameter, with two branches (3, 4), each end of which has a system (5), the said system having a center, for fixation to an apophysis (1) of a vertebra (2).

13. The interspinous prosthesis as claimed in claim 12, wherein the axis (0) of the spring is offset with respect to the straight line joining the centers of the systems for fixation to the apophyses.

14. The interspinous prosthesis as claimed in claim 12, wherein the ends of the branches (3, 4) of the spring are fastened to the apophyses (1) indirectly by way of a fastening component (5) which fixes itself to the apophyses (1).

15. The interspinous prosthesis as claimed in claim 14, wherein the fastening component (5) is a clip comprising a generally flat elongate blade whose ends are curved on the same side in order to form a hook (6, 7) for fastening to the apophysis (1) for which it is intended.

16. The interspinous prosthesis as claimed in claim 15, wherein the elongate blade has a Y shape in order to form two hooks (7) at one end and a single hook (6) at the other end.

17. The interspinous prosthesis as claimed in claim 15, wherein the blade comprises two sliding parts.

18. The interspinous prosthesis as claimed in claim 12, wherein the ends of the branches (3, 4) of the spring are fastened directly to the apophyses (1).

19. The interspinous prosthesis as claimed in claim 12, wherein the ends of the branches (3, 4) of the spring are fastened directly to the apophyses (1) and in that the end of each branch (3, 4) folds back in a loop shape in order to form a hook (6).

20. The interspinous prosthesis as claimed in claim 12, wherein the external diameter of the spring is 3 to 13 mm.

21. The interspinous prosthesis as claimed in claim 12, characterized in that the diameter of the wire from which the spring is made is 1 to 4 mm.

22. The interspinous prosthesis as claimed in claim 12, wherein the axis (0) of the spring, at rest, is offset with respect to the straight line joining the centers of the systems for fixation to the apophyses (1) by 4 to 16 mm.

23. A method for stabilizing a lumbar articulation, wherein an interspinous lumbar prosthesis comprising a torsion spring with two branches (3, 4), each end of which has a system (5) for fixation to an apophysis (1) of a vertebra (2) is fitted in place between two vertebrae.

24. A method of claim 23 wherein the two vertebrae are lumbar vertebrae.

25. A method of claim 23 wherein the branches of the spring are brought together with the aid of a suitable ancillary instrument, the ends of the branches are fixed to apophyses of two adjacent vertebrae, and the branches are released.

26. The interspinous prosthesis as claimed in claim 13, wherein the ends of the branches (3, 4) of the spring are fastened to the apophyses (1) indirectly by way of a fastening component (5) which fixes itself to the apophyses (1).

27. The interspinous prosthesis as claimed in claim 16, wherein the blade comprises two sliding parts.

28. The interspinous prosthesis as claimed in claim 13, wherein the ends of the branches (3, 4) of the spring are fastened directly to the apophyses (1).

29. The interspinous prosthesis as claimed in claim 13, wherein the ends of the branches (3, 4) of the spring are fastened directly to the apophyses (1) and in that the end of each branch (3, 4) folds back in a loop shape in order to form a hook (6).
